(19) United States
(12) Patent Application Publication
(10) Pub. No.: US 2020/0384047 A1
(43) Pub. Date: Dec. 10, 2020

(54) Method of Making a Dietary Supplement

(71) Applicant: Charlene Ashmore, Atwater, CA (US)

(72) Inventor: Charlene Ashmore, Atwater, CA (US)

(21) Appl. No.: 16/436,581

(22) Filed: Jun. 10, 2019

(51) Int. Cl. A61K 36/185; A61K 36/54; A61K 9/00

(52) U.S. Cl. CPC A61K 36/185; A61K 9/0056; A61K 36/54

(57) ABSTRACT

A method of making a dietary supplement includes the steps of mixing together, to define a mixture, refined avocado oil, ground Cannabis indica having a deactivated THC content of 15% to 21%, ground mixture of Cannabis sativa and Cannabis indica having a THC content of 20% to 30%, and ground mixture of Cannabis sativa and Cannabis indica containing a THC content of less than 6% and a CBD content of between 18% and 30%. Heating the mixture and then straining the mixture through a fine mesh strainer to define retained oil.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to health promoting supplement and more particularly pertains to a new health promoting supplement for improving the health of a person by utilizing various strains of Cannabis in combination with edible oil to facilitate recovery from and alleviate the symptoms of multiple health ailments including, for example, diabetes, kidney failure, and heart disease.

### (2) Description of Related Art Including Information Disclosed Under 37 CFR 1.97 and 1.98

The prior art relates to health promoting supplements utilizing Cannabis.

### BRIEF SUMMARY OF THE INVENTION

An embodiment of the disclosure meets the needs presented above by generally comprising the steps of mixing together, to define a mixture, refined avocado oil, ground Cannabis indica having a deactivated THC content of 15% to 21%, ground mixture of Cannabis sativa and Cannabis indica having a THC content of 20% to 30%, and ground mixture of Cannabis sativa and Cannabis indica containing a THC content of less than 6% and a CBD content of between 18% and 30%. Heating the mixture and then straining the mixture through a fine mesh strainer to define retained oil.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

Not Applicable

### DETAILED DESCRIPTION OF THE INVENTION

With reference now to the description below, a new pain relief bath salt composition embodying the principles and concepts of an embodiment of the disclosure will be described.

The method of making the dietary supplement includes the steps of grinding and mixing together:
1 ounce ground Cannabis indica having a deactivated THC content of 15% to 21%;
1 ounce ground mixture of Cannabis sativa and Cannabis indica having a THC content of 20% to 30%; and
1 ounce ground mixture of Cannabis sativa and Cannabis indica containing a THC content of less than 6% and a CBD content of between 18% and 30%.

To the above is added 16 ounces of refined avocado oil to define a mixture. Avocado oil is the oil recovered the fruit of the Persea americana plant. The mixture is then placed in the top pot of a double boiler, to prevent overheating though other heating methods may be employed, and heated for 1 hour at a temperature of 140° F. to 180° F. The mixture is then strained through a fine strainer to remove the Cannabis particles. The Cannabis particles may be pressed against the strainer to further extract any oil held by those particles. The oil recovered is defined as retained oil which is stored under refrigeration, such as at a temperature of between 33° F. and 45° F., in an airtight or otherwise closed container. The retained oil is then used as needed and typical dosages are from 3 drop to 20 drops per day from a conventional eye dropper.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A method of making an edible supplement for improving health comprising the steps of:
mixing together to define a mixture:
refined avocado oil;
ground Cannabis indica having a deactivated THC content of 15% to 21%;
ground mixture of Cannabis sativa and Cannabis indica having a THC content of 20% to 30%;
ground mixture of Cannabis sativa and Cannabis indica containing a THC content of less than 6% and a CBD content of between 18% and 30%;
heating said mixture; and
straining said mixture through a fine mesh strainer to define retained oil.

2. A method of making an edible supplement for improving health comprising the steps of:
mixing together to define a mixture:
16 ounces refined avocado oil;
1 ounce ground Cannabis indica having a deactivated THC content of 15% to 21%;
1 ounce ground mixture of Cannabis sativa and Cannabis indica having a THC content of 20% to 30%;
1 ounce ground mixture of Cannabis sativa and Cannabis indica containing a THC content of less than 6% and a CBD content of between 18% and 30%;
heating said mixture; and
straining said mixture through a fine mesh strainer to define retained oil.

3. A method of making an edible supplement for improving health comprising the steps of:
mixing together to define a mixture:
16 ounces refined avocado oil;
1 ounce ground Cannabis indica having a deactivated THC content of 15% to 21%;
1 ounce ground mixture of Cannabis sativa and Cannabis indica having a THC content of 20% to 30%;
1 ounce ground mixture of Cannabis sativa and Cannabis indica containing a THC content of less than 6% and a CBD content of between 18% and 30%;
pouring said mixture into a top pot of a double boiler having a temperature of 140° F. to 180° F. and heating the mixture for 1 hour;
straining said mixture through a fine mesh strainer to define retained oil; and
storing retained oil in an airtight container and refrigerate to between 33° F. and 45° F.
